(19) United States
(12) Patent Application Publication
(10) Pub. No.: US 2002/0103420 A1
(43) Pub. Date: Aug. 1, 2002

(54) Endoscope with alterable viewing angle

(76) Inventors: George Coleman, Rocky River, OH (US); Gary Gust, Huntington Beach, CA (US)

Correspondence Address: FISH & RICHARDSON, PC, 4350 LA JOLLA VILLAGE DRIVE, SUITE 500, SAN DIEGO, CA, 92122, US

(21) Appl. No.: 09/771,146

(22) Filed: Jan. 26, 2001

(51) Int. Cl. A61B 1/00; A61B 1/04

(52) U.S. Cl. 600/173; 600/170; 600/114

(57) ABSTRACT

An optical endoscope assembly. The assembly includes an endoscope portion which can be optical or electrical, surrounded by a sheath. The sheath includes a window pointing generally to the side, and a mirror which changes a position of incoming light. The mirror can be fixed or movable. If a fixed mirror is used, then a set of interchangeable sheaths can be used, each of which has a different mirror angle. The sheath can also be rotated once in the body to change an orientation angle from which the light is received. An image processing circuit may process the image received from the endoscope, including inverting at least a portion of the image.

### BACKGROUND

Optical endoscopes are known as devices that may be inserted into a body cavity in order to view an image of an inside of the body cavity. Typical optical endoscopes have a viewing lens at their terminus, which enables viewing areas that are generally in front of the endoscope's end portion.

### SUMMARY

The present system defines an endoscope which includes advantageous features. The endoscope includes a mirror which allows viewing from a specified direction that is not necessarily parallel with an axis of the endoscope. In one embodiment, that direction can be varied in specified ways.

### BRIEF DESCRIPTION OF THE DRAWINGS

These and other aspects will now be described in accordance with the drawings, in which:

FIGS. 1A and 1B show a fiber-optic endoscope system of a first embodiment, with FIG. 1A showing a fixed mirror embodiment, and FIG. 1B showing a movable mirror embodiment;

FIG. 2 shows an alternative embodiment which enables viewing dual directions at the same time through an endoscope;

FIG. 3 shows an embodiment including a surgical tool associated with the viewing tube;

FIG. 4 shows an embodiment in which the endoscope sheath conducts the illumination light;

FIG. 5 shows an embodiment with a viewing tube that is shortened relative to other embodiments;

FIG. 6 shows an embodiment with a camera located on the insertable portion of the scope; and

FIG. 7 shows an embodiment with a movable mirror.

### DETAILED DESCRIPTION

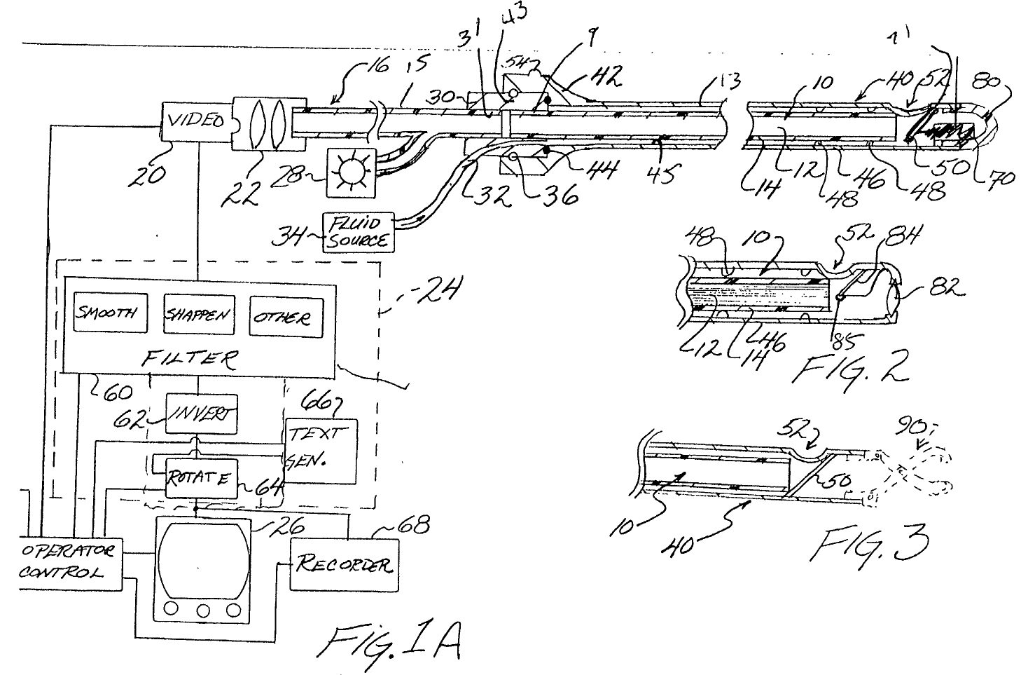

FIG. 1A shows an embodiment of the endoscope. The endoscope 10 generally includes optical fiber 12 which can be a coherent bundle of optical fibers, or an optical viewing tube or any other type of optical waveguide. An outer sheath 14 surrounds the optical fiber element 12. A space 13 is defined between the outer surface of the fiber 12, and the inner surfaces of the sheath 14. Standoff 48 may be provided between the outer surface of the fiber 12, and the inner surface 46 of the sheath. The standoffs may hold the endoscope 10 in a specified orientation within the tube, e.g., equally spaced from the inside surfaces 45 of the tube. The space 13 defines a space for irrigation fluid.

The sheath may be formed of stainless-steel or other sterilizable material. For example, sterilizable plastic may be used. The sheath also has a connector fitting 42 at an end thereof that is at the opposite end from the end where the image is acquired. As shown, the connector fitting may be an enlarged portion in which the diameter of the exterior part of the sheath becomes expanded.

A coupler 30 connects between the endoscope 10 and the extension 16. The may provide a fluid-tight but rotatable connection. In the FIG. 1A embodiment, the coupler includes an irrigation passage 32. A source of fluid 34 is connected to the irrigation passage which passes through the coupler, into the irrigation space 13. Coupler 30 also includes an attachment mechanism 36. The attachment mechanism may be an annular groove which snaps into place. The coupler may also have inner surfaces 31 which press against the outer surfaces of the endoscope section 10 and against the outer surface 15 of the extension 16. Once snapped into place, the coupler holds the sections 10 and 16 into optical registration with one another.

The connection between the sheath 14 and the extension 16 is rotatable, and also provides a fluid tight seal for irrigation fluids. In the embodiment, an oval ring 44 is received within the inner surfaces of the connector. The oval ring forms a fluid tight but rotatable seal between the sheath 14 and the remainder of the unit.

The end portion of the sheath, in operation, is adapted to be located in the area desired to be viewed. A window 52 is located at the desired area of viewing. The window can be annular, for example, and can include transparent material therein, or can be totally open. The window may also direct the irrigation fluid to the mirror in order to clean the mirror and flush the region adjacent the viewing region of the endoscope.

The area of viewing is at an angle relative to the sheath 14, which is a non-zero angle, which means that the area is not directly in front of the sheath. An optical element, e.g., mirror 50, is located adjacent the window. In this embodiment, the mirror is mounted at a fixed angle, that is, the mirror forms a fixed angle relative to an axis of the sleeve assembly. In other embodiments, the mirror may be movable as explained herein.

In this embodiment, the mirror is a fixed angle mirror—that is the mirror is mounted at a specified fixed angle. A plurality of sleeves are provided; each having a different fixed angle. The different sleeves form a set of interchangeable parts. FIG. 1A shows the mirror mounted to reflect 45 degrees, with 45 degrees being the first angle.

FIG. 1B shows an alternative portion which may be used in the embodiment of FIG. 1 and which has a different angle of reflection. In the FIG. 1A embodiment, light may be reflected by 67½ degrees. A number of different angled pieces are maintained. These pieces may allow different orientations relative to the endoscope to be viewed. Any viewing angle can be selected as is appropriate to the surgical procedure.

In operation, the user can view different angles based on the geometry of the mirror assembly which is selected. The user can also rotate the fitting portion 42 in order to view at different angular orientations relative to the fixed angle mirror.

An orientation part 54 may include an enlargement on the exterior of the fitting portion of the sleeves, and may be provided to allow tactile feedback to the operator about the viewing orientation that has been selected.

The endoscope 10 is also coupled with a video section 20. As shown, the endoscope may be coupled through the intermediate fiber length 16 to the video system 20. Video system 20 may include an optical lens assembly as well as image processing circuitry 24. Use of the optical fiber length 16 may allow the video element to be positioned more remote from the endoscope unit. In an embodiment, the insertable portion of the endoscope 10 is presterilized and packaged as a sterilized unit. The end of the extension 16 may be surface decontaminated and draped. The endoscope 10 may then be connected to the extension 16 for operation. The extension can be used many times, and with many different endoscope parts. Only the endoscope part needs total sterilization, e.g., not the whole of the extension 16. The endoscope part can be resterilized, or disposable.

The video section 20 receives light indicative of an image from the endoscope 10. The information is coupled to video processing circuitry 24 which may process the resultant video signal and generate information and/or display. The display may be sent to a monitor 26.

Image processing circuit 24 may also include a filter which can be a selectable filter which electronically smoothes the image. Different image processing operators are known in the art, and art described in (Rosenfeld, Kak textbook here) as well as in Texas Instrument application notes for its families of digital signal processors.

The mirror may also reverse the image to its mirror image. Hence, the image processor may also include an inversion part 62 to electronically mirror-invert the image in order to compensate for the effect of the mirror. The image processing may also include a rotation processor 64 which may rotate the display image. The rotation processor 64 is connected with an operator control element and enables the operator to rotate the image to a selected orientation. All of the image processing operations, including those disclosed herein and others, may be carried out by a single digital signal processor (DSP) chip, e.g. one available from Texas Instruments.

A light source 28 may direct illumination light to the area being imaged, e.g., through a portion of the fiber-optic bundle, or down a separate light guide. The illumination light is used to illuminate the area whose image is received through the endoscope 10.

A text data generator 66 may generate textual information to be displayed on the monitor 26. The textual information can include status information such as the angle of the mirror, date, time, serial numbers, patient information and the like. The video system may also include a recorder 68 which can record selected images. The recorder may be connected to the monitor 26, which is capable of providing a split screen display showing different views which occur at different times, along with textual information about those views.

FIG. 7 shows an alternative embodiment which uses a movable mirror. In this embodiment, the entire end portion 80 of the sheath may be optically clear, so that different areas can be imaged by moving the mirror. The mirror may be moved by a selectively-pressurized fluid, e.g., which is controlled by application through a syringe. The control may by via an electrically driven motor 70, as shown.

The mirror is pivoted about the pivoted mounting 71 and can be moved between its angular limits defined by the interior surfaces of the sleeve. The motor 70 may be controlled by the operator as desired until the desired angle is achieved. At any time, the motor's current position is monitored by the text generator 66, and may display an alphanumeric display of the viewing angle.

Since the mirror can be pivoted in this embodiment to image at different angles relative to the endoscope axis, and also rotated by rotation of the endoscope assembly to obtain different orientations of viewing, a very large field of view may be imaged by the single endoscope insertion. The image processor may also include image stitching software which may stitch together multiple parts obtained at different orientations or angles, to provide a single composite wide field of view.

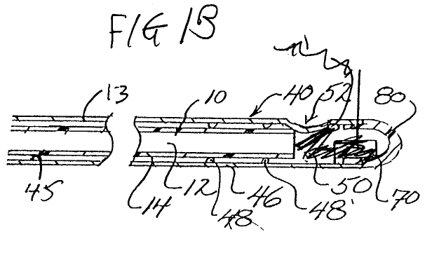

FIG. 2 shows an alternative embodiment which allows viewing multiple discontinuous views simultaneously. In this embodiment, a lens 82 is located at the front portion of the sleeve. The FIG. 2 embodiment may also include the same structure as otherwise shown in FIG. 1. Alternatively, the front of the sleeve can be left totally open in the FIG. 2 embodiment.

In this embodiment, the mirror 84 is connected to the sleeve as previously described. The mirror may be fixed as in the FIG. 1A embodiment, or may be movable as in the FIG. 7 embodiment. The mirror 84 extends over a shorter distance, however, then the corresponding mirror 50 in the FIG. 1A embodiment. In this embodiment, the mirror extends only to a point partway across the center diameter of the fiber 12. This couples the image only to part of the fiber. The other part of the fiber receives a different image from a different angle. This allows forming a split image on the fiber. A first part, e.g., half, of the image received by the fiber 12 is reflected by the mirror. This first part is obtained from the side of the fiber, at an angle defined by the angle of the mirror 84. The other part of the image is a straight ahead view which is oriented generally along the axis of the endoscope. Alternatively, another side looking view could be obtained, by using a second mirror.

The interface between the two images is at a preselected location, e.g. halfway across the fiber or some other specified percentage across the fiber. In an embodiment using a single mirror embodiment, the mirror imaging circuit 62 may be set to reverse only the corresponding fraction of the resulting image which actually comes from the mirror reflection. The mirror 84 may have a marking 85 at its edge portion to facilitate subsequent image processing. This marking may be a black line, a hologram, or any other marketing that can be found in the image field by the image processor. Markings from above the line will be inverted by the image processor and may be labeled as the first image part. Markings from below the line will not be inverted, and may be labeled as the second image part.

The mirror 84 reflects the image part such that it covers only a portion of the active area of the endoscope. The remainder of the active area of the endoscope may therefore be used for another image, or for any other purpose, such as for illumination. The ratio between the areas can be set as desired.

FIG. 3 shows another embodiment which has a surgical tool 90 attached to the outer sheath. This surgical tool may be, for example, a forceps or some kind of trocar assembly. This embodiment may use any of the other endoscope embodiments described throughout this application. In addition, the surgical tool is connected to the sleeve assembly, as previously described in the embodiments above.

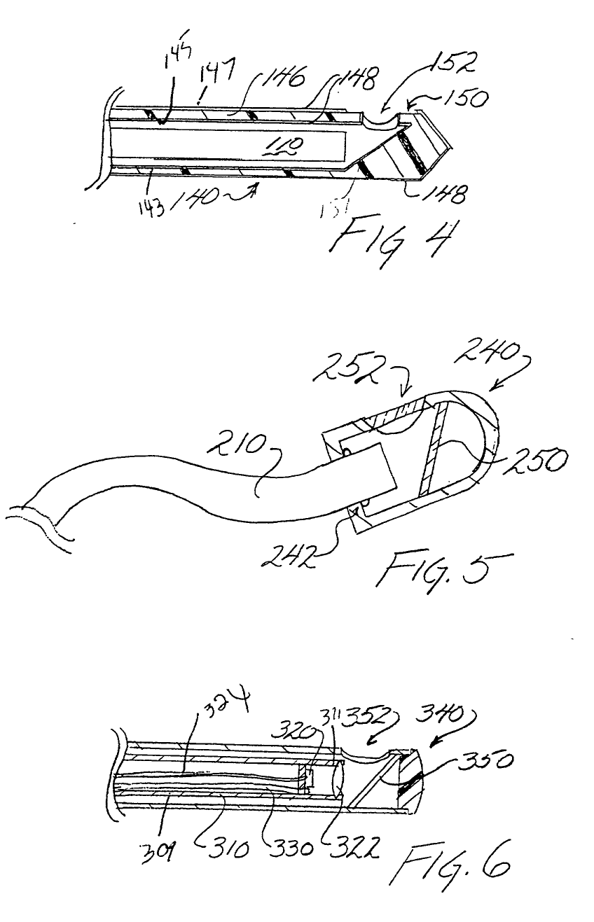

FIG. 4 shows the endoscope 110, which may be any of the endoscopes described in this application, being received in a sheath that is formed of a light transmitting material. The sheath at 146 may be tube shaped as in other embodiments. In addition, the sheath at 146 may be formed of sterilizable clear plastic. The sheath is coated on its inside and outside surfaces with a mirror or other light reflecting coating 148. The clear material 146 located between the two mirrored surfaces 148 may form an optical waveguide between the inner surface 145 and the outer surface 147. Any optical confining media may be used in place of the materials described herein. As in the other embodiments, a window 152 allows imaging of the desired area.

In operation, the illumination source 28 is optically coupled to provide its light into the optical waveguide area 145. The light travels down the optical waveguide 145, confined between the inner and outer surfaces. The light arrives at the window 152 where there is no reflective coating. This forms a ring of illumination light directed to the region adjacent the sheath. The illumination light is directed outward as shown. Reflections from the illumination light are received as an image received through the window 152, off the mirror 151, and into the endoscope 110. As in the other embodiments, the mirror can be fixed or movable, and can be available in multiple sets of different fixed angles. The sheath at 143 may also be rotated to image different areas at different orientations.

FIG. 5 shows an alternative embodiment, using a mirror sleeve assembly 240. The sleeve assembly 240 may be a shortened viewing tube relative to the other embodiments. The sleeve is received at the end of the scope section 210. The scope section 210 may include an optical fiber bundle forming a flexible light guide, leading to a video section which may be of any of the types previously described. An anchoring mechanism 242 may include a friction fit, a lip, detent arrangement, threads, bayonet fit, twist lock, or other similar sealing system. The sleeve assembly may also include an angled mirror 250 adjacent a window 252. As in the above embodiments, the mirror may be oriented at a fixed angle, with the number of different fixed angle mirrors being available as different options, or may be a movable mirror. The window may include al lens or covering shown as 254 that seals the interior of the sheath.

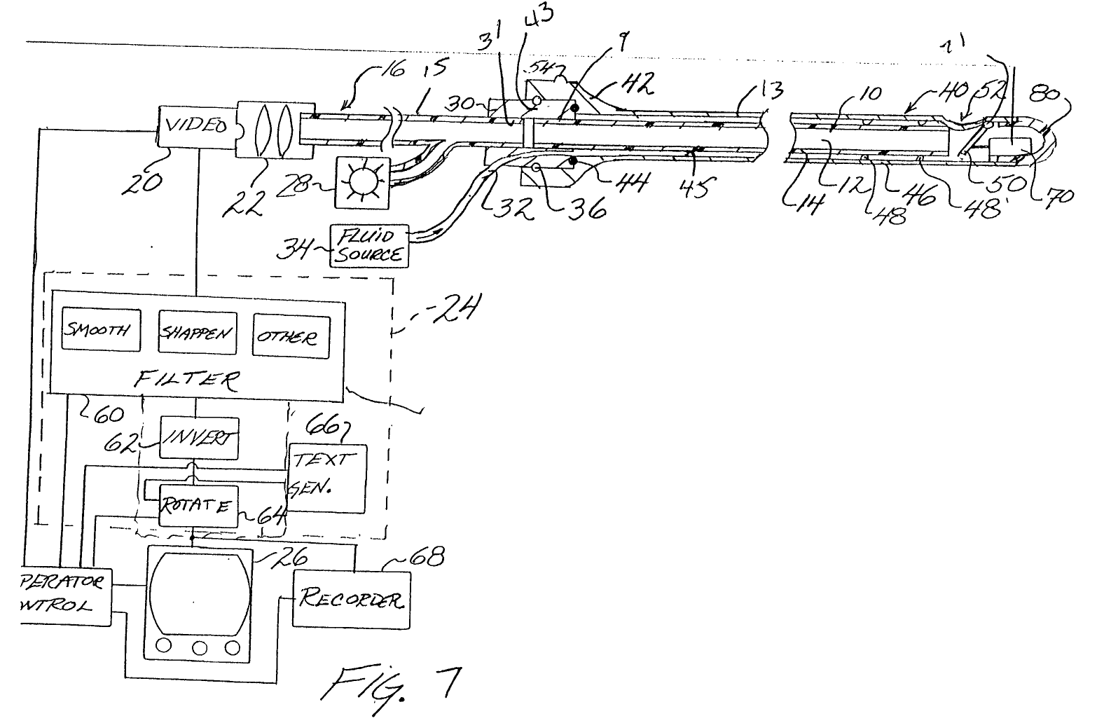

FIG. 6 shows an alternative embodiment, usable with any of the previously-described endoscopes, but which processes the image electronically, and does not use an optical cable. In this embodiment, the endoscope section 310 include walls generally shown as 309 which end in a proximal section 311. A lens 322 is attached to the end of the proximal section, and positioned and oriented to direct incoming light to a camera chip 320. The camera chip 320 accepts the incoming light, and converts the light into an electrical signal. The electrical signal is coupled to a cable 324 which extends through the wall section 309 and may connect to the video processing circuitry as previously described.

This system may also include a light guide shown as 330 extending through the scope to provide illumination light to the tip region. Alternatively, the end of the scope may include a light source, driven by electrical power sent on the cable 324 or on some other cable.

The light is preferably provided at the same angle as the imaging by the camera. The light is bounced off the mirror 350 to illuminate the area of interest. The reflections of that light also bounce off the mirror, and are received by the camera.

The lens in this, and in any of the embodiment, may be replaced by any optical element, including plain glass or a hologram, depending on the optical configuration.

This embodiment may be used with any of the previously described embodiments. For example, this embodiment may use fixed mirrors as in FIGS. 1A-1B, or a movable mirror as in FIG. 7. This may also use a partial mirror as in FIG. 2, which obtains two separate images. One of the images is coupled to a portion of the camera by the mirror 84, with the other portion of the image going to the remainder of the pixels of the camera.

This system may use any of the sleeves as previously described, and may also use the movable mirror, and also the alternative mirror configurations.

In operation, a trocar may be sheathed in a cannula and inserted through the patients skin in a region of interest. Then, the trocar is withdrawn, leading only the cannula in place as a guide. The endoscope in any of the previously-described embodiments, along with its sleeve, are then inserted as a unit through the cannula. The light source and irrigation may be started. The irrigation, if used, may provide sterile saline solution or other fluid into the area of interest. The fluid can flush debris and also clean the mirror and the area to be seen.

The angles of viewing, including the orientation angle, and the mirror angle, can then be set. The operator may rotate the mirror relative to its sleeve assembly to obtain a better view of the region of interest. In the fixed mirror embodiment, the user may remove the terminal end of the endoscope element and insert another endoscope. In one embodiment, the endoscope can be removed from the sleeve, and inserted into another sleeve with a mirror at a different fixed angle.

Although only a few embodiments have been disclosed in detail above, other modifications are possible. For example, although the above has described a separable mirror sleeve/endoscope assembly, the elements could be packaged as a single piece. Other materials besides those described herein could be used. In fact, the sheath could be made of virtually any sterilizable material. Different kinds of optical waveguides, besides the described optical fiber, can also be used.

In addition, the above has described the movable part which changes the viewing angle of the endoscope as being a mirror. Other movable components besides the mirror could be used. For example, an optical assembly such as a lens could be used which has viewing characteristics which change light position, or which change position relative to another lens. Hence, the movable component could be a movable lens assembly. In addition, holographic elements could be used, or a diffractive optical element. By moving the holographic element, a different optical characteristic is obtained. Other movable mechanisms are also contemplated.

In addition, while the above describes the signal processing being carried out using either a processor or digital signal processor, other processing techniques are also contemplated. For example, a second mirror could be used to invert the image, in place of a video processor being used for the image inversion. This second mirror can also act as a relay, which may allow different angles of light to be imaged.

The above describes a mirror being used to change the direction of light. However, other optical elements could be used for this purpose, including lenses, holographic element, diffractive optical elements or others.

All such modifications are intended to be encompassed within the following claims:

What is claimed is:

1. A system, comprising:
an endoscope section, having an image receiving portion at an area thereof, which image receiving portion receives an optical image, and transmits the optical image to another portion thereof;
a sleeve assembly, sized to cover said endoscope section and extending along an axis, and having an optical element which changes a direction of light coming from an outside said sleeve assembly, and directs light to said image receiving portion of said endoscope section from the area outside said sleeve assembly.

2. A system as in claim 1, wherein said optical element includes a mirror which forms a fixed angle relative to an axis of said sleeve assembly.

3. A system as in claim 1, further comprising an optical element moving part which allows moving an angle of said optical element relative to the axis of the sleeve assembly.

4. A system as in claim 3, wherein said optical element moving part includes a hinge element which allows moving and angle of said mirror relative to the axis of the sleeve assembly.

5. A system as in claim 3, wherein said optical element moving part includes an electrically controllable motor.

6. A system as in claim 4, wherein said optical element moving part includes a pressurizable fluid element.

7. A system as in claim 2, further comprising an additional sleeve assembly with an additional mirror that forms a different fixed angle relative to the axis of said sleeve assembly.

8. A system as in claim 2, further comprising a plurality of additional sleeve assemblies, each having a different mirror angle, forming a set.

9. A system as in claim 1, further comprising a rotatable connection between said sleeve assembly and said endoscope said endoscope section to adjust an orientation of an image being acquired.

10. A system as in claim 9, further comprising an orientation part, which is viewable from an outside of said sleeve assembly, and which indicates an orientation of rotation of said sleeve assembly.

11. A system as in claim 9, wherein said rotatable connection includes an O-ring.

12. A system as in claim 1, wherein an outer surface of said endoscope section is smaller than an inner surface of said sleeve assembly, defining a cavity between said endoscope section and said sleeve assembly.

13. A system as in claim 12, further comprising spacing elements, located in said cavity, and holdings said endoscope section at a specified orientation within said cavity.

14. A system as in claim 12, further comprising a connection to said cavity.

15. The system as in claim 14, further comprising a fluid source, connected to supply fluid to said cavity through said connection.

16. A system as in claim 12, wherein an outlet of said cavity opens near said optical element.

17. A system as in claim 15, wherein an outlet of said cavity opens near said optical element, and is located such that fluid supplied to said cavity is also supplied to said optical element.

18. A system as in claim 1, further comprising a video element, operating based on video from said endoscope section.

19. A system as in claim 18, wherein said optical element includes a mirror, and said video element electronically mirror-inverts at least a portion of an image obtained from said endoscope section.

20. A system as in claim 18, further comprising an extension cable, coupled at one end to said endoscope section and at another end to said video element.

21. A system as in claim 18, further comprising an illumination part, coupled to provide illumination to an area of imaging.

22. A system as in claim 18, wherein said video element also includes an image processing system which selectively rotates said image.

23. A system as in claim 18, wherein said video element also includes a dual display part, which simultaneously allows displaying multiple images.

24. The system as in claim 23, wherein said multiple images are images obtained at different times.

25. The system as in claim 23, wherein said multiple images are images obtained simultaneously.

26. A system as in claim 23, further comprising a text generator, which produces a textual display indicative of parameters being sensed.

27. A system as in claim 1, wherein said endoscope section is formed of an optical waveguide.

28. A system as in claim 1, wherein said endoscope section is formed of an electrical cable, and a camera receiving optical information near said image receiving portion of said endoscope.

29. A system as in claim 27, wherein said optical waveguide includes an optical fiber.

30. A system as in claim 1, wherein said endoscope section has a substantially rounded end.

31. A system as in claim 1, wherein said endoscope section has a substantially flat and.

32. A system as in claim 20, wherein said endoscope section is formed of an optical waveguide.

33. A system as in claim 32, further comprising a connector part, connecting between said endoscope section and said extension cable.

34. A system as in claim 33, wherein said connector part has inner surfaces which align said endoscope section with said extension cable.

35. A system as in claim 34, wherein said sheath has an expanded area in the vicinity of said connector part, with inner surfaces which are sized to accept said connector part.

36. A system as in claim 35, wherein said sheath is rotatable relative to said connector part.

37. A system as in claim 1, further comprising a first window portion defined in said sleeve assembly.

38. A system as in claim 1, wherein said sleeve assembly is formed of an optically non-clear material, and said window is formed to allow light to pass through said window portion in said sleeve assembly.

39. A system as in claim 37, wherein said optical element is configured to reflect light to substantially an entire part of said endoscope section.

40. A system as in claim 37, wherein said optical element is configured to reflect light to only a portion of said endoscope section.

41. A system as in claim 40, further comprising a second window portion, formed in a different area then said first window portion.

42. A system as in claim 41, wherein incoming light from said first window portion is coupled to said optical element, and incoming light from said second window portion is not coupled to said optical element.

43. A system as in claim 42, wherein said optical element includes a mirror, and said second window portion is formed in an area which is axially adjacent said endoscope section, and incoming light from said second window portion is coupled directly to said second window section without being reflected by said mirror.

44. A system as in claim 42, further comprising displaying images from both said first window portion and said second window portion.

45. A system as in claim 42, wherein said optical element includes a mirror, and further comprising an image processor that mirror-inverts said images from said first window portion, but does not mirror-invert said images from said second window portion.

46. A system as in claim 45, further comprising displaying simultaneously the images from the first window portion and from said second window portion.

47. A system as in claim 1, further comprising a surgical tool, coupled to said sheath.

48. A system as in claim 47, wherein said tool includes a forceps.

49. A system as in claim 1, further comprising an illumination element, providing illumination to an area being imaged.

50. A system as in claim 49, wherein said illumination element comprises an optical waveguide.

51. The system as in claim 49, wherein said sheath is formed of optically transparent material with reflective coatings, and said illumination is coupled to said optically transparent material.

52. A system as in claim 51, further comprising an opening in the reflective coatings in an area of the area being imaged.

53. A system as in claim 52, wherein said opening is an annular opening.

54. A system as in claim 28, further comprising an optical element, coupling optical energy to said electrical element.

55. An assembly, comprising:
an endoscope part, having a first portion adapted to receive optical energy, and a second portion adapted to supply information indicative of the optical energy;
a sheath, extending generally along an axis, and having an inner surface which is sized to be larger than an outer surface of said endoscope part, and located around said endoscope part, said sheath having an optical window located in a location which forms a predetermined non-zero degree angle with said axis, and having an optical portion located to change a direction of incoming optical energy from said optical window to the direction of said axis.

56. An assembly as in claim 55, wherein said optical element is a mirror that forms a first fixed angle relative to said axis, to thereby reflect optical energy from a specified viewing area to said optical axis.

57. An assembly as in claim 56, further comprising at least one additional sheath, having a mirror which forms a fixed angle which is different then said first fixed angle, and which can be used with said endoscope part.

58. An assembly as in claim 55, wherein said optical element is a mirror, and further comprising a pivotal mount for said mirror, allowing said mirror to be moved between different angular positions.

59. An assembly as in claim 55, further comprising a pivotal mount for said sheath, allowing said sheath to be rotated relative to said endoscope part, to receive light from a different orientation and image a different viewing area to said endoscope part.

60. An assembly as in claim 58, further comprising a pivotal mount for said sheath, allowing said sheath to be rotated relative to said endoscope part, to receive light from a different orientation and image a different viewing area to said endoscope part.

61. An assembly as in claim 55, further comprising a cavity formed in said sheath, said cavity receiving irrigation fluid.

62. An assembly as in claim 61, wherein said cavity includes an opening near said optical element, such that said irrigation fluid washes across a surface of said optical element.

63. An assembly as in claim 55, wherein said endoscope part includes an optical waveguide.

64. An assembly as in claim 55, wherein said endoscope part includes a camera, and an electrical wire receiving electrical signals from said camera.

65. An assembly as in claim 55, further comprising a video section, receiving said information indicative of the optical energy.

66. An assembly as in claim 65, wherein said video section includes an image processor which processes information indicative of the optical energy as an image.

67. An assembly as in claim 66, wherein said optical element is an element that inverts an image, and said image processor includes an image inversion element which inverts said image.

68. An assembly as in claim 55, wherein said optical element couples said incoming optical energy to only a portion of said endoscope part.

69. An assembly as in claim 68, wherein another portion of said endoscope part receives incoming optical energy indicative of another view.

70. An assembly as in claim 55, further comprising illuminating an area of viewing.

71. An assembly as in claim 70, wherein said sheath is formed of optically transparent materials, and said illuminating comprises illuminating said area via said optically transparent materials.

72. A method, comprising:
obtaining an optical image using an endoscope; and
mirror inverting at least a portion of said image.

73. A method as in claim 72, further comprising varying an angle from which said optical image is obtained.

74. A method as in claim 73, wherein said varying comprises moving a mirror to a new location to obtain said optical image from a different angle.

75. A method as in claim 73, wherein said varying comprises using a different mirror in a different fixed location to obtain said optical image from a different angle.

76. A method as in claim 72, wherein said mirror inverting comprises inverting an entire optical image.

77. A method as in claim 72, further comprising obtaining another optical image using the endoscope, and wherein said mirror inverting comprises inverting only said optical image, and not said another optical image.

78. A method as in claim 72, wherein said obtaining comprises obtaining optical energy indicative of an image, and using an optical waveguide to couple said optical image.

79. A method as in claim 72, wherein said obtaining comprises using a camera in said endoscope to obtain electrical energy indicative of an image, and using an electrical line to couple said electrical energy indicative of said image.

80. A method as in claim 72, wherein said obtaining comprises obtaining a view from an angle relative to an axis of said endoscope, and using a mirror to reflect said view in a direction of said axis.

81. A method as in claim 80, wherein said mirror reflects said image in a way that covers an entire active area of said endoscope.

82. A method as in claim 80, wherein said mirror reflects said image in a way that covers only a part of an entire active area of said endoscope.

83. A method as in claim 82, further comprising obtaining another image using the active area of said endoscope other than said part of said active area.

84. A method as in claim 83 wherein said endoscope includes an optical fiber, and said mirror reflects said image to only a portion of said optical fiber.

85. A method as in claim 72, further comprising providing illumination for a view obtained by said endoscope.

86. A method as in claim 72, further comprising allowing rotation of an area of imaging.

87. A method, comprising:
inserting an endoscope into a body cavity;
first obtaining an image from said endoscope from a specified viewing area in said body cavity; and
without removing said endoscope from said body cavity, second obtaining an image of a different viewing area than said specified viewing area.

88. A method as in claim 87, wherein said first obtaining an image comprises obtaining an image from a direction that makes a specified nonzero angle with an axis of said endoscope.

89. A method as in claim 88, wherein said second obtaining comprises rotating said endoscope in said body cavity to orient to a different angle.

90. A method as in claim 87, wherein said obtaining comprises using a mirror to reflect an image from a direction that makes a nonzero angle with an axis of said endoscope, to a direction of said axis of said endoscope.

91. A method as in claim 87, wherein said second obtaining comprises moving an internal component of said endoscope to change a viewing angle.

92. A method as in claim 91, wherein said component is a mirror.

93. A method as in claim 91, wherein said moving comprises actuating an electronic motor to move said component.

94. A method as in claim 88, wherein said second obtaining comprises moving a component of said endoscope to change said nonzero angle and thereby view a different viewing area.

95. A method as in claim 94, wherein said component of said endoscope which is moved is a component which is internal to said endoscope.

96. A method as in claim 94, wherein said component is a mirror.

97. A method as in claim 88, wherein said second obtaining comprises either rotating said endoscope in said body cavity to orient to a different angle, and/or moving a component of said endoscope to change an effective viewing angle by changing an angle of an optical path being imaged by said endoscope.

98. A method as in claim 97, wherein said component of said endoscope is an internal component which is moved to change said optical path.

99. A method as in claim 97, wherein said component is a mirror.

100. A method, comprising:
using an endoscope to obtain an optical image from a body cavity of a patient; and
varying an angle from which said optical image is obtained.

101. A method as in claim 99, further comprising varying an angle relative to an axis of said endoscope, from which said optical image is obtained.

102. A method as in claim 100, wherein said varying comprises moving a mirror to a new location to obtain said optical image from a different angle.

103. A method as in claim 100, wherein said varying comprises using a different mirror in a different fixed location to obtain said optical image from a different angle.

104. A method as in claim 100, wherein said varying comprises moving a movable optical element to a different location which reflects optical energy at a different angle.

105. A method as in claim 100, wherein said optical image is obtained in the form of optical energy, and is guided on a light waveguide in said endoscope.

106. A method as in claim 105, wherein said light waveguide is a fiber-optic cable.

107. A method as in claim 100, wherein said optical image is obtained in the form of electrical energy, and is guided on an electrical cable in said endoscope.

108. A method, comprising:
first obtaining a first image from a first position in a body cavity; and
second obtaining, using the same device as used to obtain said first image, and simultaneously in time to receiving said first image, a second image from a second position in the same body cavity.

109. A method as in claim 108, further comprising image processing said first image and said second image.

110. A method as in claim 109, wherein said image processing comprises image processing said first image in a different way than image processing in said second image.

111. A method as in claim 110, wherein said image processing in said first image includes mirror-inverting said first image, and said image processing in said second image does not include mirror-inverting said second image.

112. A method as in claim 108, wherein said first and second obtaining comprises obtaining an image from the first position and applying the image from the first position to a first portion of an image acquisition element, and said second obtaining comprises obtaining the image from the second position, and applying this image from the second position to a second portion of the image acquisition element, different than the first portion of the image acquisition element.

113. A method as in claim 112, where the image acquisition element includes a light waveguide.

114. A method as in claim 112, where the image acquisition element includes an electronic camera.

115. An endoscope, comprising:
a scope portion, which extends in a first direction, and which includes an image coupling element for acquiring an image and coupling said image in said first direction, said scope portion formed with a window which is positioned to acquire an image from a direction that makes a nonzero angle with said first direction; and
an optical direction changing element, which changes a direction of said image from said direction, to the first direction.

116. An endoscope as in claim 115, wherein said image coupling element includes an optical waveguide.

117. An endoscope as in claim 116, wherein said optical waveguide includes an optical fiber.

118. An endoscope as in claim 115, wherein said image coupling element includes an electronic camera and a cable carrying an electrical signal from said electronic camera.

119. An endoscope as in claim 115, wherein said direction changing element includes a mirror.

120. An endoscope as in claim 119, wherein said mirror includes a hinging element, and is movable relative to said hinging element.

121. An endoscope, comprising:
a scope portion, having a first window adapted to acquire an image of a first viewing area from a first direction, and a second window adapted to acquire an image of a second viewing area from a second direction, different than said first direction; and
an image element, simultaneously acquiring said images from said first and second viewing areas.

122. An endoscope as in claim 121, wherein said image element includes an optical waveguide.

123. An endoscope as in claim 121, wherein said image element includes an electrical camera.

124. An endoscope as in claim 121, further comprising a direction changing element which changes an angle of said image from said first direction.

125. An endoscope as in claim 124, wherein said direction changing element includes a movable element which changes a direction of a light path.

126. An endoscope as in claim 124, wherein said direction changing element includes a hinged portion coupled to said scope portion.

127. An endoscope as in claim 124, wherein said scope portion includes a mirror at a fixed angle, and said direction changing element includes a separate portion of said scope portion which includes a mirror at a different fixed angle.

128. An endoscope as in claim 121, further comprising an image processor, which image processes said image of said first viewing area in a different way than image processing of said image of said second viewing area.

129. An endoscope as in claim 128, wherein said different way comprises inverting said image of said first viewing area.

130. A method, comprising:
an endoscope portion including an optical coupling element and a sheath covering said optical coupling element; and
an image processing element, receiving an image from said optical coupling element, and processing said image to invert at least a portion of said image.

131. An apparatus as in claim 130, wherein said image processing element also selectively rotates said image.

132. An apparatus as in claim 130, wherein said image processing element also adds text to said image, said text indicative of conditions of imaging.

133. An apparatus as in claim 130, wherein said sheath includes an optical element which changes an angle of incidence of incoming light.

134. A method, comprising:
using an optical endoscope with a sheath to obtain an image from a specified nonzero angle of incidence relative to said endoscope; and
changing a sheath to use a different another sheath that images from a different angle of incidence, and then using said optical endoscope to obtain a second image from a second specified nonzero angle of incidence.

135. A method as in claim 135, further comprising illuminating said image using a same optical path as is used for said imaging, to illuminate said image at any angle of incidence being currently used.

136. An endoscope, comprising:
an optical receiving element, and
an optical endoscope system obtaining an image of a specified area, and coupling said image to only a portion of said optical receiving element, a rest of said optical receiving element being used for a purpose other than obtaining said image of said specified area.

137. An endoscope as in claim 136, wherein said purpose is for obtaining another image, different than said image of said specified area.
